(19) United States
(12) Patent Application Publication
(10) Pub. No.: US 2015/0273022 A1
(43) Pub. Date: Oct. 1, 2015

(54) STABILIZED ULTRA-RAPID-ACTING INSULIN FORMULATIONS

(71) Applicant: Biodel Inc., Danbury, CT (US)

(72) Inventors: Bryan R. Wilson, Brewster, NY (US); Pragati Ravula, Ridgefield, CT (US); Ming Li, Yorktown Heights, NY (US); Roderike Pohl, Sherman, CT (US); Robert Hauser, Columbia, MD (US)

(21) Appl. No.: 14/618,619

(22) Filed: Feb. 10, 2015

Related U.S. Application Data
(60) Provisional application No. 61/938,012, filed on Feb. 10, 2014.

(51) Int. Cl. A61K 38/28; A61K 47/18; A61K 33/06; A61K 47/02; A61K 47/22; A61K 9/00; A61K 47/12

(52) U.S. Cl. CPC A61K 38/28; A61K 9/0019; A61K 47/183; A61K 47/12; A61K 47/02; A61K 47/22; A61K 33/06

(57) ABSTRACT

Compositions and methods for enhancing the stability of rapid acting injectable insulin formulations have been developed for subcutaneous injection. The formulations contain insulin in combination with a zinc chelator such as ethylenediaminetetraacetic acid (“EDTA”), a dissolution/stabilization agent such as citric acid, a magnesium salt, a zinc compound and, optionally, additional excipients. New presentations include rapid acting concentrated insulin formulations and a way to enhance the absorption of commercially available rapid acting analog formulations while maintaining insulin stability.

### CROSS-REFERENCE TO RELATED APPLICATIONS

This application claims benefit of and priority to U.S. Ser. No. 61/938,012 filed on Feb. 10, 2014, which is incorporated herein by reference.

### FIELD OF THE INVENTION

The invention is in the general field of injectable rapid acting drug delivery insulin formulations and methods of their use and reduction of pain on injection.

### BACKGROUND OF THE INVENTION

Diabetes is a disease characterized by abnormally high levels of blood glucose and inadequate levels of insulin. There are two major types of diabetes—Type 1 and Type 2. In Type 1 diabetes, the body produces no insulin. In the early stages of Type 2 diabetes, although the pancreas does produce insulin, either the body does not produce the insulin at the right time or the body's cells ignore the insulin, a condition known as insulin resistance.

Even before any other symptoms are present, one of the first effects of Type 2 diabetes is the loss of the meal-induced first-phase insulin release. In the absence of the first-phase insulin release, the liver will not receive its signal to stop making glucose. As a result, the liver will continue to produce glucose at a time when the body begins to produce new glucose through the digestion of the meal. As a result, the blood glucose level of patients with diabetes goes too high after eating, a condition known as hyperglycemia.

Because patients with Type 1 diabetes produce no insulin, the primary treatment for Type 1 diabetes is daily intensive insulin therapy. The treatment of Type 2 diabetes typically starts with management of diet and exercise. Although helpful in the short-run, treatment through diet and exercise alone is not an effective long-term solution for the vast majority of patients with Type 2 diabetes. When diet and exercise are no longer sufficient, treatment commences with various non-insulin oral medications. However, because of the limitations of non-insulin treatments, many patients with Type 2 diabetes deteriorate over time and eventually require insulin therapy to support their metabolism.

Insulin therapy has certain limitations. For example, even when properly administered, insulin injections do not replicate the natural time-action profile of insulin. In particular, the natural spike of the first-phase insulin release in a person without diabetes results in blood insulin levels rising within several minutes of the entry into the blood of glucose from a meal. By contrast, injected insulin enters the blood slowly, with peak insulin levels occurring within 80 to 100 minutes following the injection of regular human insulin. The 1990's saw the introduction of rapid-acting insulin analogs, such as HUMALOG® (insulin lispro), NOVOLOG® (insulin aspart) and APIDRA® (insulin glulisine). However, even with the rapid-acting insulin analogs, peak insulin levels typically occur within 50 to 70 minutes following the injection.

Insulin formulations with an even more rapid onset of action, such as VIAject®, are described in U.S. Pat. No. 7,279,457, and U.S. Published Applications 2007/0235365, 2008/0085298, 2008/90753, and 2008/0096800, and Steiner, et al., Diabetologia, 51:1602-1606 (2008). The rapid onset of VIAject® results from the inclusion of two key excipients, a zinc chelator such as disodium EDTA (EDTA) and/or calcium disodium EDTA which rapidly dissociates insulin hexamers into monomers and dimers and a dissolution/stabilization agent such as citric acid which stabilizes the dissociated monomers and dimers prior to being absorbed into the blood (Pohl et al, J. Diabetes Sci. and Technology, 2012. 6(4)755-763).

Early clinical trials with this product showed injection site discomfort. Inclusion of calcium, either as calcium chloride and/or the calcium salt of the EDTA, decreased injection site pain. However, the addition of calcium altered the pharmacokinetics. Replacing the calcium with Magnesium reduced the injection site pain but the insulin formulations but did not have acceptable stability and shelf life.

It is an object of this invention to provide compositions of ultra-rapid acting injectable insulin compositions with reduced injection site discomfort and improved stability.

It is also an object of the present invention to provide specific concentrated insulin formulations for treating insulin resistant diabetic which modulate the pharmacokinetics and pharmacodynamics of injectable insulin compositions by increasing the rate of absorption from the site of subcutaneous injection.

### SUMMARY OF THE INVENTION

Insulin formulations with rapid onset of action, improved injection site tolerability, and improved stability been developed. The formulations are based on a selection of excipients in amounts effective to enhance the absorption of commercially available or formulated rapid acting analog formulations while maintaining insulin stability and having acceptable injection site pain.

The formulations contain insulin in combination with a zinc chelator such as ethylenediamine tetraacetic acid (“EDTA”), preferably the sodium and/or calcium salt thereof, one or more dissolution/stabilization agent such as citric acid and/or sodium citrate, one or more magnesium compounds, a zinc compound and, optionally, additional excipients such as preservatives and pH buffers.

Preferred formulations typically contain 100, 200 or 400 IU/ml insulin and the insulin is preferably an insulin analog or regular human insulin.

The concentration of the zinc chelator, for example, disodium EDTA, can range from 0.1 to 5 mg/mL and citrate can be included at a concentration between 0.6 mg/ml and 4.8 mg/ml of the formulation.

The concentration of magnesium compounds is from about 0.1 to about 10 mg/ml, preferably from about 0.1 to about 5 mg/ml, more preferably from about 0.1 to about 2 mg/ml, most preferably from about 0.2 to about 2 mg/ml.

A total zinc concentration greater than 0.01 mg/ml can be included in the formulations, preferably between 0.01 and 0.065 mg/ml (U-100 analog andU-200, U-300 and U-400) can be included in the formulations In this embodiment, the zinc to insulin hexamer ratio is preferably greater than 0.3 more preferably between 0.5 and 2.6, and most preferably, between 0.5 and 0.9.

The formulation can also include a nicotinic compound in a range between 25 mM and 250 mM. Preferably, the formulation contains about 50 mM nicotinamide. When present, sodium citrate is in an amount between 0.5 and 4.8 mg/ml.

Methods of controlling blood glucose levels in a subject include administering the insulin formulations disclosed herein to a subject in need thereof. In the preferred embodiment, the formulations are administered via subcutaneous injection.

### BRIEF DESCRIPTION OF THE DRAWINGS

FIGS. 1A and 1B are bar graphs showing the percent potency of insulin (lispro) (FIG. 1A) and high molecular weight (“HMWP”) insulin protein as measured using the USP standard assay (FIG. 1B) in HUMALOG® (insulin lispro), BIOD-238 and BIOD-250.

FIGS. 2A and 2B show the loss of potency of insulin (lispro)(IU) (FIG. 2A), and gain in percent HMWP (FIG. 2B) in BIOD-238, BIOD-250, BIOD-286, and BIOD-288.

FIGS. 3A to 3F show dynamic light scattering analysis performed on the following formulations: HUMALOG®, BIOD-250, and BIOD-290. The baseline measurements are shown in FIGS. 3A (HUMALOG®), 3C (BIOD-250), and 3E (BIOD-290), which were compared to the measurements at 37° C. at day 7, shown in FIGS. 3B (HUMALOG®), 3D (BIOD-250), and 3F (BIOD-290).

FIGS. 4A to 4F show ultracentrifugation data for HUMALOG® (FIGS. 4A and 4B), BIOD-250 (FIGS. 4C and 4D), and BIOD-290 (FIGS. 4E and 4F).

FIG. 5A shows the Thioflavin T fluorescence over time for NOVOLOG® (insulin aspart), HUMALOG®, HUMULIN® (recombinant human insulin) 100 and HUMULIN® 500. FIGS. 5B and 5C show kinetic Fibril Formation Profile in a Thioflavin T Assay, of Lots of BIOD-238 and BIOD-250 Vs. NOVOLOG®, HUMULIN® U-100, HUMULIN® R U-500 and HLTMALOG® (FIG. 5B); and BIOD-290 vs. NOVOLOG®, HUMULIN® U-100, and HUMALOG® (FIG. 5C).

FIGS. 6A and 6B show the blood insulin or glucose levels as a function of time in diabetic swine, following administration of different insulin formulations: HUMALOG®, BIOD-250, BIOD-290 and BIOD-294 (FIGS. 6A, pharmacokinetics). The blood glucose levels (baseline subtracted) are shown in FIGS. 6B.

FIG. 7 is a line graph showing insulin absorption for BIOD-300 and NOVOLOG®.

FIGS. 8A and 8B show the stability of insulin as measured by insulin potency, as a function of time, in response to Zinc:hexamer ratios of 0 (BIOD-300), 0.4, 0.5, 0.6, 0.7, 0.8 and 0.9 in formulations made from Novolog (8A) or insulin aspart (8B).

### DETAILED DESCRIPTION OF THE INVENTION

### I. Definitions

As used herein, “insulin” refers to human or non-human, recombinant, purified or synthetic insulin or insulin analogues, unless otherwise specified.

As used herein, “Human insulin” is the human peptide hormone secreted by the pancreas, whether isolated from a natural source or made by genetically altered microorganisms. As used herein, “non-human insulin” is the same as human insulin but from an animal source such as pig or cow.

As used herein, an insulin analogue is an altered insulin, different from the insulin secreted by the pancreas, but still available to the body for performing the same action as natural insulin. Through genetic engineering of the underlying DNA, the amino acid sequence of insulin can be changed to alter its ADME (absorption, distribution, metabolism, and excretion) characteristics. Examples include insulin lispro, insulin glargine, insulin aspart, insulin glulisine, and insulin detemir. The insulin can also be modified chemically, for example, by acetylation. As used herein, human insulin analogues are altered human insulin which is able to perform the same action as human insulin.

As used herein, a “chelator” or “chelating agent”, refers to a chemical compound that has the ability to form one or more bonds to zinc ions. The bonds are typically ionic or coordination bonds. The chelator can be an inorganic or an organic compound. A chelate complex is a complex in which the metal ion is bound to two or more atoms of the chelating agent.

As used herein, a “solubilizing agent”, is a compound that increases the solubility of materials in a solvent, for example, insulin in an aqueous solution. Examples of solubilizing agents include surfactants such as polysorbates (TWEEN®); solvents such as ethanol; micelle forming compounds, such as oxyethylene monostearate; and pH-modifying agents.

As used herein, a “dissolution/stabilization agent”—is an acid or a salt thereof that, when added to insulin and EDTA, enhances the transport and absorption of insulin relative to HCl and EDTA at the same pH. HCl is not a dissolution/stabilization agent but may aid in solubilization. Citric acid is a dissolution/stabilization agent.

As used herein, “inorganic magnesium compound” or “inorganic magnesium salt” refers to compounds in which the anion does not contain one or more carbon atoms.

As used herein, “organic magnesium compound” or “organic magnesium salt” refers to compounds in which the anion contains one or more carbon atoms.

As used herein, an “excipient” is an inactive substance other than a chelator or dissolution/stabilization agent, used as a carrier for the insulin or used to aid the process by which a product is manufactured. In such cases, the active substance is dissolved or mixed with an excipient.

As used herein, a “physiological pH” is between 6.8 and 7.6, preferably between 7 and 7.5, most preferably about 7.4.

As used herein, “Cmax” is the maximum or peak concentration of a drug observed after its administration.

As used herein, “Tmax” is the time at which maximum concentration (Cmax) occurs. As used herein, ½ Tmax is the time at which half maximal concentration (½ Cmax) of insulin occurs in the blood. This may also be expressed as T50% earlymax.

As used herein, “Improved/Enhanced insulin stability” refers to loss of insulin potency of less than 5 IU at 7 days at 37° C. and/or HMWP under/below 2%, more preferably under/below than 1.7%, and even more preferably, under/below 1.5% in the same time frame and temperature.

As used herein, “Insulin stability” refers to loss of insulin potency and/or increase in quantity of high molecular weight insulin protein (HMWP) Insulin stability can be monitored using several high pressure liquid chromatography (HPLC) assays that determine the insulin potency, quantity of high molecular weight protein (HMWP), and quantity of other insulin breakdown products such as oxidated or deamidated insulin.

Zinc (Zn): Hexamer ratio is used herein interchangeably with Zinc (Zn): insulin Hexamer.

### II. Formulations

Formulations include insulin or an insulin analog, a zinc chelator and a dissolution/stabilizing agent(s), a zinc compound, optionally, and one or more other excipients. Insulin in the formulations disclosed herein show a rapid onset of action, improved injection site tolerability, and improved stability, as measured by insulin potency and HMWP. Preferably, insulin formulations which show improved stability maintain insulin potency at 95% at 7 days (37° C.) and/or HMWP under 2%, more preferably less than 1.7%, and even more preferably, below 1.5% over the same time frame and temperature. Stability is enhanced as a function of the concentration of an agent selected from the group consisting of zinc chelator, zinc compound, dissolution/stabilization agent, or combinations thereof. Preferably, the enhanced insulin formulations (containing a zinc chelator, dissolution/stabilizing agent(s), a zinc compound) have a zinc to insulin hexamer ratio greater than 0.3 more preferably between 0.5 and 2.6, and most preferably, between 0.5 and 0.9. A preferred zinc compound is zinc oxide. In some embodiments, the stability of insulin is enhanced when compared to BIOD-238 and/or BIOD-250 formulations disclosed herein.

A magnesium salt has been found to not significantly alter the pharmacokinetic profile while at the same time decreasing the injection site pain. In some embodiments, the formulation can include one or more magnesium compounds such as magnesium EDTA, Mg(OH)₂, MgSO₄, or combinations thereof. M-cresol can be added for its anti-microbial properties and enhancement of shelf life.

The formulations are preferably suitable for subcutaneous administration and are rapidly absorbed into the subcutaneous tissue. The choice of dissolution/stabilization agent and chelator, the concentration of both the dissolution/stabilization agent and the chelator, and the pH that the formulation is adjusted to, all have a profound effect on the efficacy of the system. While many combinations have efficacy, the preferred embodiment is chosen for reasons including safety, comfort, stability, regulatory profile, and performance.

In the preferred embodiment, at least one of the formulation ingredients is selected to mask charges on the insulin. This is believed to facilitate the transmembrane transport of the insulin and thereby increase both the onset of action and bioavailability for the insulin. The ingredients are also selected to form compositions that dissolve rapidly in aqueous medium. Preferably the insulin is absorbed and transported to the plasma quickly, resulting in a rapid onset of action, preferably beginning within about 5 minutes following administration and peaking at about 15-30 minutes following administration.

The chelator, such as EDTA, chelates the zinc within the insulin, thereby removing the zinc from the insulin hexamer. This causes the hexameric insulin to dissociate into its dimeric and monomeric forms and retards reassembly into the hexameric state post injection. Since these two forms exist in a concentration-driven equilibrium, as the monomers are absorbed, more monomers are created. Thus, as insulin monomers are absorbed through the subcutaneous tissue, additional dimers dissemble to form more monomers. The monomeric form has a molecular weight that is less than one-sixth the molecular weight of the hexameric form, thereby markedly increasing both the speed and quantity of insulin absorption. To the extent that the chelator (such as EDTA) and/or dissolution/stabilization agent (such as citric acid) hydrogen bond with the insulin, it is believed that they mask the charge on the insulin, facilitating its transmembrane transport and thereby increasing both the onset of action and bioavailability of the insulin.

A. Insulin

The concentration of the insulin in the formulation varies from 100-500 units/mL, more preferably, between 100-400 units/ml. Preferred formulations typically contain 100, 200 or 400 IU/ml insulin.

In some embodiments, insulin is the only pharmaceutically active agent or bioactive peptide in the formulation. In these embodiments, the formulation does not include other peptides which modify the release kinetics of insulin from the formulation, for example hyaluronan degrading enzymes, or other hypoglycemic peptides.

Insulins which can be added to the formulations disclosed herein include, but are not limited to fast acting insulins, rapid acting insulin, concentrated insulins, intermediate acting insulins, long acting insulins or combinations thereof. In some embodiments the insulin analog is an analog obtained through genetic engineering of the underlying DNA, which changes the amino acid sequence of insulin and alters its ADME (absorption, distribution, metabolism, and excretion) characteristics.

Fast acting insulins are intended to respond to the glucose derived from ingestion of carbohydrates during a meal. Fast acting insulins start to work within one to 20 minutes, peaking about one hour later and lasting from three to five hours. Fast acting insulin takes about two hours to fully absorb into the systemic circulation. Fast acting insulins include regular recombinant human insulin (such as HUMULIN®, marketed by Eli Lilly, and NOVALIN®, marketed by Novo Nordisk A/S) which are administered in an isotonic solution at pH 7. Bovine and porcine insulins, which differ in several amino acids to human insulin, but are bioactive in humans, are also fast acting insulins. Recombinant human insulin is available from a number of other sources. The dosages of the insulin depend on its bioavailability and the patient to be treated. Insulin is generally included in a dosage range of 1.5-200 IU, depending on the level of insulin resistance of the individual. Typically, insulin is provided in 100 IU vials, though other presentations of 200, 400 or 500 U/ml are described herein. In the most preferred embodiment the injectable formulation is a volume of 1 ml containing 100 U of insulin. Additional embodiments include higher concentration insulin formulations, the most preferred being U-400.

Concentrated forms of insulin are provided for insulin resistant individuals. The commercially available formulation HUMULIN® R U-500 has a very long duration of action and is suitable for basal use only due to its slow release profiles.

Rapid-acting insulin that have been modified or have altered locations of amino acids in order to enhance their rate of absorption. Commercially available rapid acting insulins include insulin lispro (Lysine-Proline insulin, sold by Eli Lilly as HUMALOG®), insulin glulisine (sold by Sanofi-Aventis as APIDRA®) and insulin aspart (sold by Novo Nordisk as NOVOLOG®).

Intermediate-acting insulin has a longer lifespan than short-acting insulin but it is slower to start working and takes longer to reach its maximum strength. Intermediate-acting insulin usually starts working within 2-4 hours after injection, peaks somewhere between 4-14 hours and remains effective up to 24 hours. Types of intermediate-acting insulin include NPH (Neutra) Protamine Hagedorn) and LENTE insulin. NPH insulin contains protamine which slows down the speed of absorption so that the insulin takes longer to reach the bloodstream but has a longer peak and lifespan. Intermediate acting insulins may be combined with rapid acting insulins at neutral pH, to reduce the total number of injections per day.

Combinations of rapid acting insulin and NPH insulin are commercially available to fulfill the need for prandial and basal use in a single injection. These include regular recombinant insulin based insulin combinations (HUMULIN® 70/30 (70% human insulin isophane and 30% human insulin, Eli Lilly) or analog based insulin combinations, such HUMALOG®Mix 75/25 (75% insulin lispro protamine suspension and 25% insulin lispro solution) (Eli Lilly).

Examples of long acting insulins that can be included in the formulations disclosed herein are insulin glargine (marketed under the tradename LANTUS®, Sanofi Aventis) and insulin detemir (LEVEMIR®, Novo Nordisk A/S).

B. Dissolution/Stabilization Agents and Zinc Chelators

Certain polyacids and zinc chelators enhance insulin uptake and transport. The chelator binds the zinc holding the monomers together to form a hexamer, dissociating the hexamer into the monomeric or dimeric form and facilitating absorption of the insulin into the tissues surrounding the site of administration (e.g. mucosa, or fatty tissue). The polyacids appear to mask charges on the dissociated insulin monomer/dimers, and to stabilize the dissociated monomers and dimers. In addition, the chelator hydrogen may bond to the insulin, thereby aiding the charge masking of the insulin monomers and facilitating transmembrane transport of the insulin monomers.

In general the ratio of citric acid/sodium citrate to disodium EDTA is in the range of 5:1 to 40:1, preferably about 21:1.

i. Dissolution/Stabilization Agent

Acids which are effective as dissolution/stabilization agents include acetic acid, ascorbic acid, citric acid, glutamic acid, aspartic acid, succinic acid, fumaric acid, maleic acid, adipic acid, and salts thereof, relative to hydrochloric acid, which is not a charge masking agent. The effective acids are all diacids or polyacids. Preferred dissolution/stabilization agents are citric acid and/or sodium citrate. Hydrochloric acid may be used for pH adjustment, in combination with any of the formulations, but is not a dissolution/stabilization agent.

The acid may be added directly or in the form of a salt, which dissociates in aqueous solution. Salts of the acids include sodium acetate, ascorbate, citrate, glutamate, aspartate, succinate, fumarate, maleate, and adipate. Salts of organic acids can be prepared using a variety of bases including, but not limited to, metal hydroxides, metal oxides, metal carbonates and bicarbonates, metal amines, as well as ammonium bases, such as ammonium chloride, ammonium carbonate, etc. Suitable metals include monovalent and polyvalent metal ions. Exemplary metals ions include the Group I metals, such as lithium, sodium, and potassium; Group II metals, such as barium, magnesium, calcium, and strontium; and metalloids such as aluminum. Polyvalent metal ions may be desirable for organic acids containing more than one carboxylic acid group since these ions can simultaneously complex to more than one carboxylic acid group. The preferred dissolution/stabilization agent when the insulin formulation has a pH in the physiological pH range is sodium citrate. The preferred concentration for a dissolution agent is in the between 0.6 mg/ml and 4.8 mg/ml citrate. Some embodiments include between 0.6 and 2.4 mg/ml citrate.

(ii) Chelators

Chelators that may be used with the insulin formulations disclosed herein include ethylenediaminetetraacetic acid (EDTA), EGTA, alginic acid, alpha lipoic acid, dimercaptosuccinic acid (DMSA), CDTA (1,2-diaminocyclohexanetetraacetic acid), and trisodium citrate (TSC). Hydrochloric acid is used in conjunction with TSC to adjust the pH, and in the process gives rise to the formation of citric acid, which is a dissolution/stabilization agent.

In the preferred embodiment, the chelator is EDTA. In the most preferred embodiment, the formulation contains insulin, disodium EDTA, and a dissolution/stabilization agent such as citric acid or sodium citrate and magnesium sulfate

A range of 2.42×10⁻⁴ M to 9.68×10⁻²M EDTA corresponds to a weight/volume of about 0.07 mg/ml to about 28 mg/ml if the EDTA is Ethylenediaminetetraacetic acid with a molar mass of approximately 292 grams/mole. The formulations preferably contain disodium EDTA. For U-100 analog formulations, the range is 0.01 to 2 mg/ml disodium EDTA; more preferably 0.06 to 0.5. For concentrated U-200, 300 and 400 analog and RHI formulations the range is 0.01 to 5 mg/mL EDTA, more preferably 0.06 to 4 mg/mL disodium EDTA.

In some preferred embodiments, the amount of EDTA is equal to or less than 0.2 mg/ml. The amount of insulin can be between 0.1125 and 0.225 mg/ml, more preferably, about 0.1125 mg/ml. For example, the formulations can include 100 or 200 IU/ml lispro and 0.1125 mg/ml disodium EDTA, 2.4 mg/ml sodium citrate, 4 mM MgSO4, 16 mg glycerol, with m-cresol and phosphate. In other embodiments preferred formulations include 400 IU/ml insulin, 0.225 mg/ml disodium EDTA, 4.8 mg/ml sodium citrate, optionally, 4 mM MgSO4, 16 mg glycerol, with m-cresol and phosphate.

C. Zinc Compounds

A total zinc concentration greater than 0.01 mg/ml can be included in the formulations, preferably between 0.01 and 0.065 mg/ml (U-100 analog and U-200, U-300 and U-400). In this embodiment, the zinc to insulin hexamer ratio is preferably greater than 0.3 more preferably between 0.5 and 2.6, and most preferably, between 0.5 and 0.9. A preferred zinc compound is zinc oxide.

D. Magnesium Compounds

The formulations contain one or more pharmaceutically acceptable magnesium compounds. As discussed above, EDTA can cause irritation at the injection site due to the complexation of endogenous calcium at the site of administration. While the inclusion of calcium EDTA can ameliorate this irritation, the addition of calcium EDTA to the formulation slows down the insulin absorption. In order to minimize or prevent injection site irritation and not change the rate of subcutaneous absorption, one or more magnesium compounds are incorporated into the formulation.

The magnesium compounds can be an inorganic and/or organic magnesium salt. Suitable magnesium inorganic salts include, but are not limited to, magnesium hydroxide (Mg(OH)₂), magnesium sulfate Mg(SO₄), magnesium halides, such as magnesium chloride (MgCl₂), magnesium bromide (MgBr₂), and magnesium iodide (MgI₂); magnesium pyrophosphate, magnesium sulfate heptahydrate, and magnesium oxide (MgO₂).

Suitable magnesium organic salts include, but are not limited to, magnesium EDTA, magnesium lactate, amino acid chelates, such as magnesium aspartate; magnesium acetate, magnesium carbonate (Mg(CO₃)₂), magnesium citrate, and magnesium gluconate.

In particular embodiments, the one or more magnesium compounds is magnesium EDTA, Mg(OH)₂, MgSO₄, or combinations thereof. In the preferred embodiment, the magnesium compound is MgSO₄.

The concentration of the one or more magnesium compounds is from about 0.1 to about 10 mg/ml, preferably from about 0.1 to about 5 mg/ml, more preferably from about 0.1 to about 2 mg/ml, most preferably from about 0.2 to about 2 mg/ml. For example, the formulations contain about 0.2-0.3 mg/ml Mg(OH)₂ (e.g., 0.282 mg/mL), about 1.7-3.0 mg/mL magnesium EDTA (e.g., 1.89 mg/mL), and/or about 0.1-1.5 mg/mL magnesium sulfate (e.g., 0.481 or 0.985 mg/mL). A preferred magnesium compound is MgSO₄.

Excipients

Pharmaceutical compositions may be formulated in a conventional manner using one or more physiologically acceptable carriers comprising excipients and auxiliaries which facilitate processing of the active compounds into preparations which can be used pharmaceutically. Fonnulation of drugs is discussed in, for example, Hoover, John E., Remington's Pharmaceutical Sciences, Mack Publishing Co., Easton, Pa. (1975), and Liberman, H.A. and Lachman, L., Eds., Pharmaceutical Dosage Forms, Marcel Decker, New York, N.Y. (1980).

In the preferred embodiment, one or more solubilizing agents are included with the insulin to promote rapid dissolution in aqueous media. Suitable solubilizing agents include wetting agents such as polysorbates, glycerin and poloxamers, non-ionic and ionic surfactants, food acids and bases (e.g. sodium bicarbonate), and alcohols, and buffer salts for pH control. In a preferred embodiment the pH is adjusted using hydrochloric acid (HCl) or sodium hydroxide (NaOH). The pH of the injectable formulation is typically between about 6.8-7.8, in some embodiments between 6.8 and 7.5, or between 6.8 and 7.2, and in some embodiments, greater than 7.0. More preferably the pH is about 7.1 or 7.2.

Stabilizers are used to inhibit or retard drug decomposition reactions which include, by way of example, oxidative reactions. A number of stabilizers may be used. Suitable stabilizers include buffers; such as citrates, phosphates and acetates; polysaccharides, such as cellulose and cellulose derivatives, sulfated polysaccharides complex and simple alcohols, such as glycerol (or glycerin, or glycerine); bacteriostatic agents such as phenol, benzyl alcohol, meta-cresol (m-cresol), 2-phenoxyethanol and methyl/propyl paraben; isotonic agents, such as sodium chloride, glycerol (or glycerin/glycerine), cyclic amino acids, amino acids and glucose; lecithins, such as example natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins. Also, solvent or co-solvent systems (ethanol, PEG-300, glycerin, propylene glycol) and solubilizing agents such as polysorbates 20/80; poloxamer 188 and sorbitol. In one example, the stabilizer may be a combination of glycerol, bacteriostatic agents and isotonic agents. The most preferred formulations include glycerin and m-cresol. The range for glycerin is about 1-35 mg/ml, preferably about 10-25 mg/ml, most preferably about 19.5-22.5 mg/ml. The range for m-cresol is about 0.75-6 mg/ml, preferably about 1.8-3.2 mg/ml, most preferably about 2 or 3 mg/ml. Calcium chloride can be added to the formulation to “neutralize” any free EDTA and sodium citrate and/or citric acid is added to stabilize the dissociated monomer. Calcium chloride is more typically added to the formulation when the chelator is disodium EDTA. It is added in matched approximately equimolar concentration to the disodium EDTA. The effective range is 80-120% of disodium EDTA. A further possible candidate for this is magnesium, added in similar quantities.

In some embodiments, commercial preparations of insulin and insulin analogs preparations can be used as the insulin of the formulations disclosed herein. Therefore, the final formulation can include additional excipients commonly found in the commercial preparations of insulin and insulin analogs, including, but not limited to, zinc, zinc chloride, phenol, sodium phosphate, zinc oxide, disodium hydrogen phosphate, sodium chloride, tromethamine, and polysorbate 20. These may also be removed from these commercially available preparations prior to adding the chelator and dissolution/stabilizing agents described herein.

### III. Methods of Making the Formulations

In a preferred embodiment, the injectable formulation contains insulin, disodium and/or calcium disodium EDTA, citric acid, saline or glycerin, m-Cresol and magnesium salt. In the most preferred embodiment, the subcutaneous injectable formulation is produced by combining water, disodium EDTA, magnesium salt such as MgSO₄, citric acid, glycerin, m-Cresol and insulin by sterile filtration into multi-use injection vials or cartridges.

Methods of making the injectable insulin formulations are described in detail in the Examples below.

In one embodiment, the EDTA is added to the formulation(s) prior to the citric acid. In another embodiment, sodium citrate is added instead of citric acid. In the preferred embodiment, citric acid is added to the formulation(s) prior to the EDTA. In one preferred embodiment, the components of the formulation are added to water: citric acid, EDTA, glycerin, m-Cresol, magnesium salt and insulin. Glycerol and m-Cresol are added as a solution while citric acid, EDTA and magnesium salt may be added as powder, crystalline or pre-dissolved in water

In some embodiments, the subcutaneous injectable formulation is produced by mixing water, citric acid, EDTA, glycerin and m-Cresol to forma solution (referred to as the “diluent”) which is filtered and sterilized. The insulin is separately added to water, sterile filtered and a designated amount is added to a number of separate sterile injection bottles which is then lyophilized to form a powder. The lyophilized powder is stored separately from the diluent to retain its stability. Prior to administration, the diluent is added to the insulin injection bottle to dissolve the insulin and create the final reconstituted product.

In another embodiment, the insulin is in solution and the excipients are lyophilized, spray dried, and added to the insulin prior to injection. In another embodiment, the excipients are made as a concentrated liquid and introduced to the liquid insulin prior to injection.

After the predetermined amount of insulin is subcutaneously injected into the patient, the remaining insulin solution may be stored, preferably with refrigeration. In a preferred embodiment, the insulin is prepared as an aqueous solution at about pH 7.0, in vials or cartridges and kept at 4° C.

### IV. Methods of Using Formulations

The formulations may be injected subcutaneously or intramuscularly. The formulation is designed to be rapidly absorbed and transported to the plasma for systemic delivery.

Formulations containing insulin as the active agent may be administered to type 1 or type 2 diabetic patients before or during a meal. Due to the rapid absorption, the compositions can shut off the conversion of glycogen to glucose in the liver, thereby preventing hyperglycemia, the main cause of complications from diabetes and the first symptom of type 2 diabetes. Currently available, standard, subcutaneous injections of human insulin must be administered about one half to one hour prior to eating to provide a less than desired effect, because the insulin is absorbed too slowly to shut off the production of glucose in the liver. These new ultrarapid acting formulations may be taken closer to the meal. A potential benefit to this formulation with enhanced pharmacokinetics may be a decrease in the incidence or severity of obesity that is a frequent complication of insulin treatment.

### EXAMPLES

The stability profiles of different insulin formulations were evaluated under accelerated testing conditions of 37° C. with the minimum requirement being that the insulin stay within specifications (i.e., potency remaining at 95% and HMWP less than 1.5%) for 37° C. for 7 days, with the intent of having at least 18 months stability at 5° C. Insulin lispro potency and High Molecular Weight Protein (HMWP) were the two primary stability measurements utilized in the screening program. The accelerated testing condition of 37° C. was chosen to enable the rapid screening for stable formulations under the assumption that the relative degradation profiles versus the analog (HUMALOG®) at 37° C. would provide a reliable indication of the relative stability at the commercially relevant conditions of 5° C. cold storage followed by 25° C. (room temperature during in-use period) or 30° C. (pump usage).

### Example 1

### Insulin Lispro Potency and Formation of HMWP in Different Insulin Formulations

The stability of insulin was tested using two insulin formulations (BIOD-238 and BIOD-250), which have been previously shown to have injection site pain comparable to HUMALOGs.

Materials and Methods

Each milliliter of HUMALOG® contains: insulin lispro (100 IU), 16 mg glycerin, 1.88 mg dibasic sodium phosphate, 3.15 mg Metacresol, zinc oxide content adjusted to provide 0.0197 mg zinc ion, and trace amounts of phenol.

Each milliliter of BIOD-238 contains: insulin lispro (100 IU), 0.225 mg of Na₂EDTA, 2.4 mg of sodium citrate, 16.0 mg of glycerin, 3.15 mg of m-cresol as a preservative, 0.1 mg phenol, 1.88 mg of disodium phosphate and 0.0197 mg of ZnO.

Each milliliter of BIOD-250 contains: insulin lispro (100 IU), 0.45 mg of Na₂EDTA, 2.4 mg of sodium citrate, 16.0 mg of glycerin, 3.15 mg of in-cresol as a preservative, 0.1 mg phenol, 1.88 md of disodium phosphate, 0.0197 mg of ZnO and 0.481 mg of MgSO₄ (4 mM).

Insulin stability was monitored using high pressure liquid chromatography (HPLC) assay to determine the insulin potency and size exclusion chromatography to determine the quantity of high molecular weight protein.

Results

The potency of insulin lispro and formation of HMWP in BIOD-238 and BIOD-250 is shown in Table 1.

TABLE 1
Potency of insulin lispro and formation of HMWP in BIOD-238
and BIOD-250 following storage at 5° C. and 25° C.

| | BIOD-238 | | BIOD-250 | |
|---|---|---|---|---|
| | 5° C. | 25° C. | 5° C. | 25° C. |
| Insulin Potency | 14 months | 22 days | 23 months | 23 days |
| HMWP | 11 months | 43 days | 8 months | 44 days |

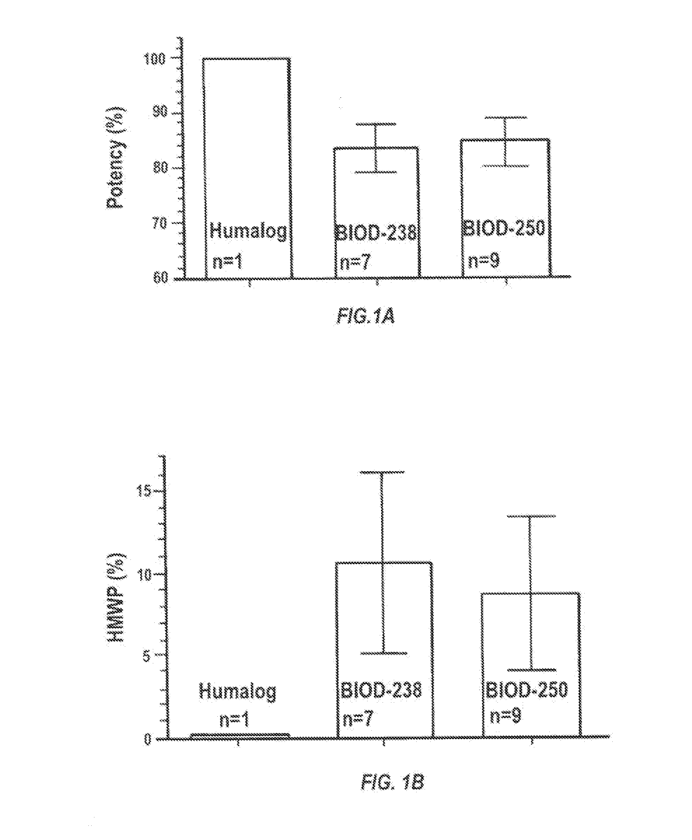

The potency of insulin lispro in HUMALOG®, BIOD-238 and BIOD-250 following storage at 37° C. for 7 days, is shown in FIG. 1A. Formation of HMWP in HUMALOG®, BIOD-238 and BIOD-250 following storage at 37° C. for 7 days is shown in FIG. 1B.

Based on this data, the potency of the test formulations at 5° C. should maintain 95-105 IU for least 18-24 months and the HMWP should remain under 1.5% over the same time frame. In this case the HMWP was out of specification by 11 or 8 months.

### Example 2

### Effect of Zinc Chelator Concentration on the Stability of Insulin Lispro and the Formation of HMWP

The aim of this study was to evaluate the stability of insulin lispro as a function of changing concentrations of zinc chelator. In these studies, 4 different insulin formulations (BIOD-238, BIOD-250, BIOD-288 and BIOD-286) were studied, with varying concentrations of EDTA.

Methods and Materials

The contents of BIOD-238 and BIOD-250 are provided above.

Each milliliter of BIOD-286 contains: 100 U/ml insulin lispro (˜3.86 mg), 0.1125 mg disodium EDTA, 4 mM MgSO4, 2.4 mg of sodium citrate, 0.0231 mg/ml of ZnO.

Each milliliter of BIOD-288 contains: 100 U/mL insulin lispro (˜3.86 mg/ml), 0.1125 mg disodium EDTA 2.4 mg of sodium citrate, 0.0194 mg of ZnO, 4 mM MgSO4.

By contrast with respect to EDTA, BIOD 238 contains 0.225 mg/ml of Na₂EDTA, and BIOD-250 contains 0.45 mg of Na₂EDTA.

Results:

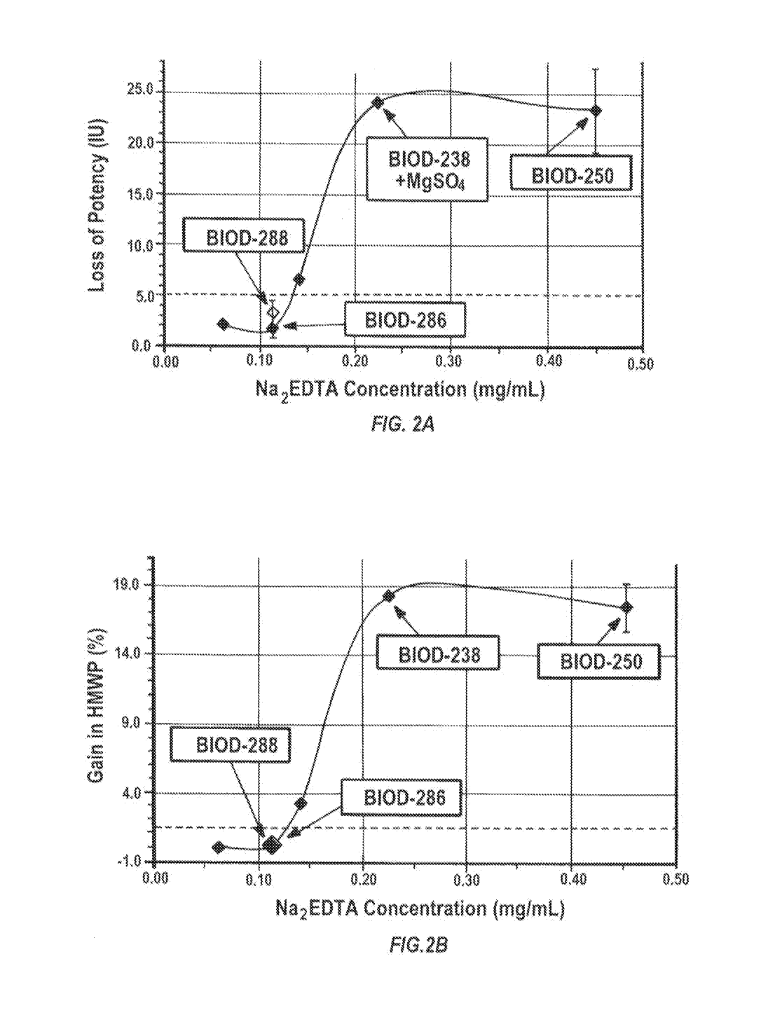

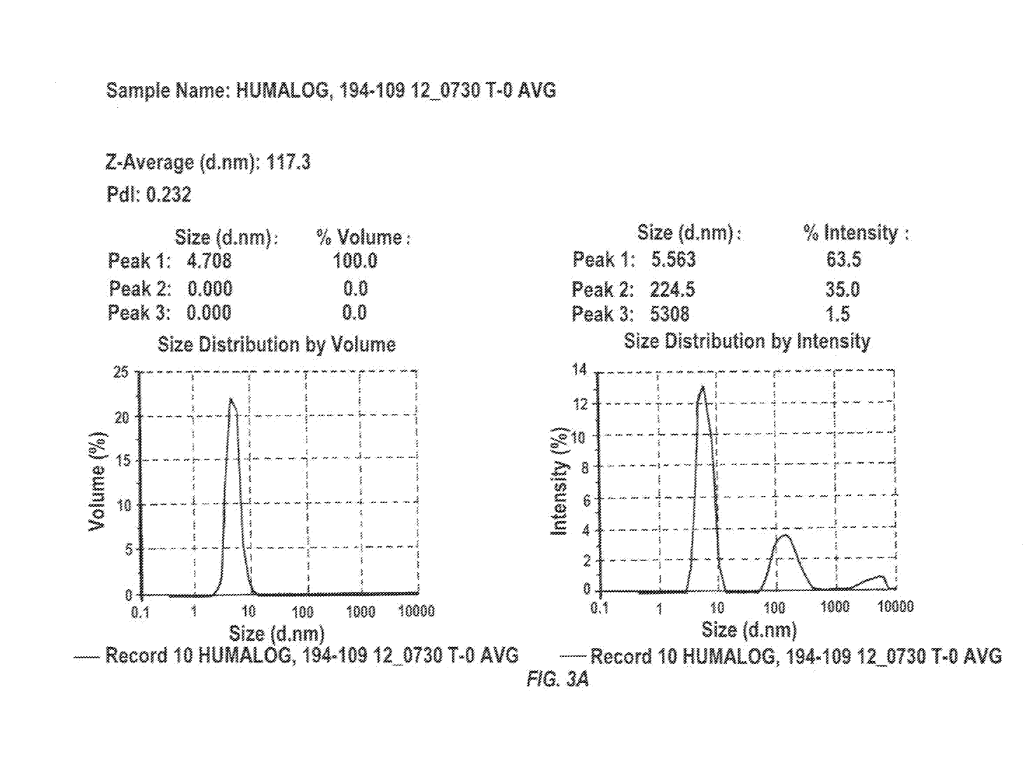

As shown in FIGS. 2A and 2B, reducing the concentration of EDTA (from 0.45 mg/ml (BIOD-250)) to 0.1125 mg/ml improves stability of insulin lispro, as measured by loss of potency of insulin lispro (FIG. 2A) and gain in HMWP (FIG. 2B) each, at 37° C. for 7 days.

### Example 3

### The Effect of Zinc Concentration (and Ratio of Zinc Pairs:Hexamer) on Insulin Lispro Potency and Formation of HMWP

Materials and Methods

Formulations were prepared with a fixed EDTA concentration of 0.1125 mg/ml. Zinc oxide levels were varied at 0.0124 mg/ml, 0.016 mg/ml and 0.0197 mg/ml (the concentration of zinc oxide in HUMALOG). The formulations used in these experiments are shown in Table 2. Insulin potency and the presence of HMWP were determined as previously described.

TABLE 2
Insulin Lispro formulations with varying concentrations of zinc oxide, providing different ratios of zinc pairs:lispro hexamer

| | | | | | | | mM of total | 7 days at 37° C. | |
|---|---|---|---|---|---|---|---|---|---|
| | EDTA | Citrate | Lispro | Zn Conc. | Ratio Zn | Zn | Zinc net of | API loss | HMWP |
| Code, NB# | (mg/mL) | (mg/mL) | (mg/mL) | (mg/mL) | pairs:hexamer | (mM) | EDTA bound Zn | (IU) | gain (%) |
| BIOD-239.01.01 | 0.1125 | 2.4 | HUMALOG ® | 0.0197 | 0.0031 | 0.303 | 6.576E−04 | 8.123 | 3.953 |
| BIOD-286.07 | 0.1125 | 2.4 | 3.86 | 0.0269 | 0.5030 | 0.413 | 1.110E−01 | 8.767 | 3.908 |
| BIOD-288, | 0.1125 | 2.4 | 3.86 | 0.0298 | 0.7043 | 0.458 | 1.555E−01 | 3.501 | 0.231 |
| n = 4 | | | | | | | | | |
| BIOD-286, | 0.1125 | 2.4 | 3.86 | 0.0327 | 0.9112 | 0.504 | 2.011E−01 | 1.689 | 0.061 |
| n = 10 | | | | | | | | | |

Results

The data shows that increasing the zinc/“ratio of zinc pairs to Lispro hexamer” improves stability as measured by change in potency and decrease in HMWP.

### Example 4

### The Effect of Citrate Concentration on Insulin Lispro Potency and Formation of HMWP

Formulations were prepared with a fixed EDTA concentration of 0.1125 mg/ml and zinc ion concentration of 0.0197 mg/ml. Citrate levels were varied at 0.6 mg/ml, 1.2 mg/ml and 2.4 mg/ml. The formulations used in these experiments are shown in Table 3.

TABLE 3
Formulations of insulin lispro with varying concentrations of citrate

| | | | | | | mM Zn | mM of total | 14 days at 37° C. | |
|---|---|---|---|---|---|---|---|---|---|
| | EDTA | Citrate | HUMALOG | Zn Conc. | Ratio Zn | (mol. wt | Zinc net of | API loss | HMWP |
| Code, NB# | (mg/mL) | (mg/mL) | (IU/mL) | (mg/mL) | pairs:hexamer | 65 g/mol) | EDTA bound Zn | (IU) | % gain |
| BIOD-239.01.01 | 0.1125 | 2.4 | 100 | 0.0197 | 0.0031 | 0.303 | 6.576E−04 | 19.75 | 11.81 |
| BIOD-239.02.01 | 0.1125 | 1.2 | 100 | 0.0197 | 0.0031 | 0.303 | 6.576E−04 | 12.97 | 6.23 |
| BIOD-239.03.01 | 0.1125 | 0.6 | 100 | 0.0197 | 0.0031 | 0.303 | 6.576E−04 | 4.97 | 1.48 |

Results Reducing the citrate levels in an insulin lispro formulation improves stability as measured by change in potency and decrease in HMWP.

### Example 5

### Interaction Between “Ratio of Zinc Pairs to Lispro Hexamer” and Citrate Concentration

Formulations were prepared with a fixed EDTA level of 0.1125 mg/mL. Citrate levels varied at 0.6 mg/mL, 1.2 mg/mL or 2.4 mg/mL. Zinc levels varied at 0.413 mM, 0.458 mM or 0.504 mM. These formulations are shown in Table 4. The formulations were placed in an accelerated degradation study. For these studies, formulations were held at 37° C. and assayed for potency and HMWP at 7 and 14 days. The results are shown in Tables 5A and 5B.

TABLE 4
Insulin formulations with varying concentrations of zinc oxide and citrate

| | | | | | | | mM of total | |
|---|---|---|---|---|---|---|---|---|
| | EDTA | Citrate | Lispro | Zn Conc. | Ratio Zn | Zn conc | Zinc net of | mM |
| Code, NB# | (mg/mL) | (mg/mL) | (mg/mL) | (mg/ml) | pairs:hexamer | (mM) | EDTA bound Zn | Citrate |
| BIOD-286.07 | 0.1125 | 2.4 | 3.86 | 0.027 | 0.503 | 0.413 | 1.110E−01 | 8.16 |
| BIOD-239.02 | 0.1125 | 1.2 | 3.86 | 0.027 | 0.503 | 0.413 | 1.110E−01 | 4.08 |
| BIOD-239.03 | 0.1125 | 0.6 | 3.86 | 0.027 | 0.503 | 0.413 | 1.110E−01 | 2.04 |
| BIOD-288 | 0.1125 | 2.4 | 3.86 | 0.030 | 0.704 | 0.458 | 1.555E−01 | 8.16 |
| BIOD-239.02 | 0.1125 | 1.2 | 3.86 | 0.030 | 0.704 | 0.458 | 1.555E−01 | 4.08 |
| BIOD-239.03 | 0.1125 | 0.6 | 3.86 | 0.030 | 0.704 | 0.458 | 1.555E−01 | 2.04 |
| BIOD-286 | 0.1125 | 2.4 | 3.86 | 0.033 | 0.911 | 0.504 | 2.011E−01 | 8.16 |
| BIOD-239.02 | 0.1125 | 1.2 | 3.86 | 0.033 | 0.911 | 0.504 | 2.011E−01 | 4.08 |
| BIOD-239.03 | 0.1125 | 0.6 | 3.86 | 0.033 | 0.911 | 0.504 | 2.011E−01 | 2.04 |
| BIOD-239.01.01 | 0.1125 | 2.4 | Humalog ® | 0.0197 | 0.003 | 0.303 | 6.576E−04 | 8.16 |
| BIOD-239.02.01 | 0.1125 | 1.2 | Humalog ® | 0.0197 | 0.003 | 0.303 | 6.576E−04 | 4.08 |
| BIOD-239.03.01 | 0.1125 | 0.6 | Humalog ® | 0.0197 | 0.003 | 0.303 | 6.576E−04 | 2.04 |

Results.

TABLE 5A
Loss of Potency at 37° C., measured after 14 days with
varying citrate and Ratio of Zinc: lispro Hexamer

| Ratio Zn: | Citrate Level (mg/mL) | | |
|---|---|---|---|
| Hexamer | 0.6 | 1.2 | 2.4 |
| 0.003 | 4.972 | 12.968 | 19.753 |
| 0.503 | 5.963 | 9.745 | — |
| 0.704 | 2.781 | 3.089 | 3.335 |
| 0.911 | 0.156 | −0.647 | 2.462 |

TABLE 5B
Gain in HMWP at 37° C., measured after 14 days with
varying citrate and Ratio of Zinc: lispro Hexamer

| Ratio Zn: | Citrate Level (mg/mL) | | |
|---|---|---|---|
| Hexamer | 0.6 | 1.2 | 2.4 |
| 0.003 | 1.477 | 6.232 | 11.807 |
| 0.503 | 1.061 | 3.647 | — |
| 0.704 | 0.061 | 0.133 | 0.539 |
| 0.911 | 0.199 | 0.118 | 0.122 |

Conclusion: Low ratios of Zn:hexamer produce more HMWP. Reducing the concentration of citrate reduces the formation of HMWP at Zn:hexamer ratios less than 0.9. This reduction of HMWP is not seen with Zn:hexamer ratios of 0.9 and greater.

### Example 6

### Enhancement of Insulin Absorption Using Nicotinamide

Nicotinamide has been identified as a molecule that may act as an absorption enhancer for ultra-rapid acting insulin (URAI) formulations. The structure of nicotinamide is shown below.

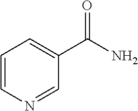

Insulin formulations including nicotinic acid or nicotinamide are disclosed in U.S. Pat. No. 5,382,574. Nicotinamide was added to insulin formulations to enhance absorption. These studies also evaluated the effect (if any) of different concentrations of nicotinamide on short term accelerated insulin stability (14 days at 37° C.).

Methods and Materials

50 mM or 100 mM of Nicotinamide was added to Lispro formulations containing ratios of Zn pairs to Lispro hexamers of 0.9, 0.8, 0.7 or 0.6. The formulations tested are shown in Table 6.

TABLE 6
Insulin Formulations containing Nicotinamide

| | | | | | Ratio of | | mM of total | | Nicotin- | 7 days at 37° C. | |
|---|---|---|---|---|---|---|---|---|---|---|---|
| | EDTA | Citrate | Lispro | Zn Oxide | Zn pairs | Zn | Zinc net of | mM | amide | API | HMWP |
| Code: NB# | (mg/mL) | (mg/mL) | (mg/mL) | (mg/mL) | to hexamer | (mM) | EDTA bound Zn | Citrate | (mM) | (IU loss) | (% gain) |
| BIOD-286* | 0.1125 | 2.4 | 3.788 | 0.0231 | 0.9112 | 0.501 | 1.98E−01 | 8.1605 | 0 | — | — |
| BIOD-294 | 0.1125 | 2.4 | 3.788 | 0.0231 | 0.9112 | 0.501 | 1.98E−01 | 8.1605 | 50 | 2.138 | 0.061 |
| BIOD-286.11 | 0.1125 | 2.4 | 3.788 | 0.0231 | 0.9112 | 0.501 | 1.98E−01 | 8.1605 | 100 | 2.536 | 0.065 |
| BIOD-286.09 | 0.1125 | 2.4 | 3.788 | 0.0231 | 0.9112 | 0.501 | 1.98E−01 | 8.1605 | 250 | 1.277 | 0.133 |
| BIOD-286.08 | 0.1125 | 2.4 | 3.788 | 0.0211 | 0.8000 | 0.476 | 1.74E−01 | 8.1605 | 0 | 0.358 | 0.066 |
| BIOD-286.08.01 | 0.1125 | 2.4 | 3.788 | 0.0211 | 0.8000 | 0.476 | 1.74E−01 | 8.1605 | 50 | 3.467 | 0.118 |
| BIOD-286.08.02 | 0.1125 | 2.4 | 3.788 | 0.0211 | 0.8000 | 0.476 | 1.74E−01 | 8.1605 | 100 | 3.932 | 0.138 |
| BIOD-290, | 0.1125 | 2.4 | 3.788 | 0.0194 | 0.7043 | 0.456 | 1.53E−01 | 8.1605 | 50 | 2.081 | 0.101 |
| BIOD-288.02 | 0.1125 | 2.4 | 3.788 | 0.0194 | 0.7043 | 0.456 | 1.53E−01 | 8.1605 | 100 | 0.695 | 0.096 |
| BIOD-288.03 | 0.1125 | 2.4 | 3.788 | 0.0176 | 0.6000 | 0.433 | 1.30E−01 | 8.1605 | 50 | 0.880 | 0.109 |
| BIOD-289.01 | 0.1125 | 0.6 | 3.788 | 0.0176 | 0.6000 | 0.433 | 1.30E−01 | 2.0401 | 100 | 2.075 | 0.161 |

*A different batch of 100 IU lispro than described above

Results:

Generally the greater ratios of Zn pairs to Lispro hexamers resulted in a lower 1-1MWP gain. There was no effect on stability with concentration levels up to 100 mM Nicotinamide.

The effect of varying concentrations of nicotinamide (25 mM; 50 mM, 75 mM and 100 mM) on insulin stability is shown in Table 7.

TABLE 7
Effect of Nicotinamide on insulin stability at 37° C. for 7 and 14 days

| | | | | Ratio | | 7 days at 37° C. | | 14 days at 37° C. | |
|---|---|---|---|---|---|---|---|---|---|
| | EDTA | Citrate | Lispro | Zn pairs | Nicotin- | API | HMWP | API | HMWP |
| Code: NB# | (mg/mL) | (mg/mL) | (mg/mL) | to hexamer | amide | Loss (IU) | Gain (%) | Loss (IU) | Gain (%) |
| BIOD-288*: | 0.1125 | 2.4 | 3.788 | 0.7043 | — | 3.199 | 0.187 | 4.621 | 0.589 |
| n = 4 | | | | | | | | | |
| BIOD-288.05: | 0.1125 | 2.4 | 3.788 | 0.7043 | 25 mM | 2.176 | 0.210 | 4.585 | 0.490 |
| 236-049 | | | | | | | | | |
| BIOD-290: | 0.1125 | 2.4 | 3.788 | 0.7043 | 50 mM | 2.687 | 0.198 | 4.603 | 0.540 |
| n = 3 | | | | | | | | | |
| BIOD-293: | 0.1125 | 2.4 | 3.788 | 0.7043 | 75 mM | 2.134 | 0.125 | 4.373 | 0.543 |
| BIOD-288.02 | 0.1125 | 2.4 | 3.788 | 0.7043 | 100 mM | 0.695 | 0.096 | 4.937 | 0.879 |

*A different batch of 100 IU lispro than described above

The addition of nicotinamide to the formulations did not have an adverse effect on their stability.

A summary of the composition of candidate formulations identified as a result of the studies above is provided in Table 8 below.

TABLE 8
Candidate Formulations

| | BIOD-286 | BIOD-288 | BIOD-290 | BIOD-294 |
|---|---|---|---|---|
| Component | mg/mL | mg/mL | mg/mL | mg/mL |
| Insulin Lispro | 3.788 | 3.788 | 3.788 | 3.788 |
| API (Activity = | | | | |
| 26.4 U/mg) | | | | |
| Zinc Oxide | 0.0231 | 0.0194 | 0.0194 | 0.0231 |
| (additional) | | | | |
| Phenol | 0.026 | 0.026 | 0.026 | 0.026 |
| m-Cresol | 3.150 | 3.150 | 3.150 | 3.150 |
| Glycerol | 16.00 | 16.00 | 16.00 | 16.00 |
| Sodium | 1.880 | 1.880 | 1.880 | 1.880 |
| monophosphate | | | | |
| Trisodium citrate | 2.400 | 2.400 | 2.400 | 2.400 |
| Disodium EDTA | 0.1125 | 0.1125 | 0.1125 | 0.1125 |
| Nicotinamide | — | — | 6.106 | 6.106 |
| MgSO₄ (4 mM) | 0.481 | 0.481 | 0.481 | 0.481 |

### Example 8

### Physical Characterization Studies to Determine the Mechanism of Stabilization Action and Ultra-Rapid-Acting Characteristics by Dynamic Light Scattering

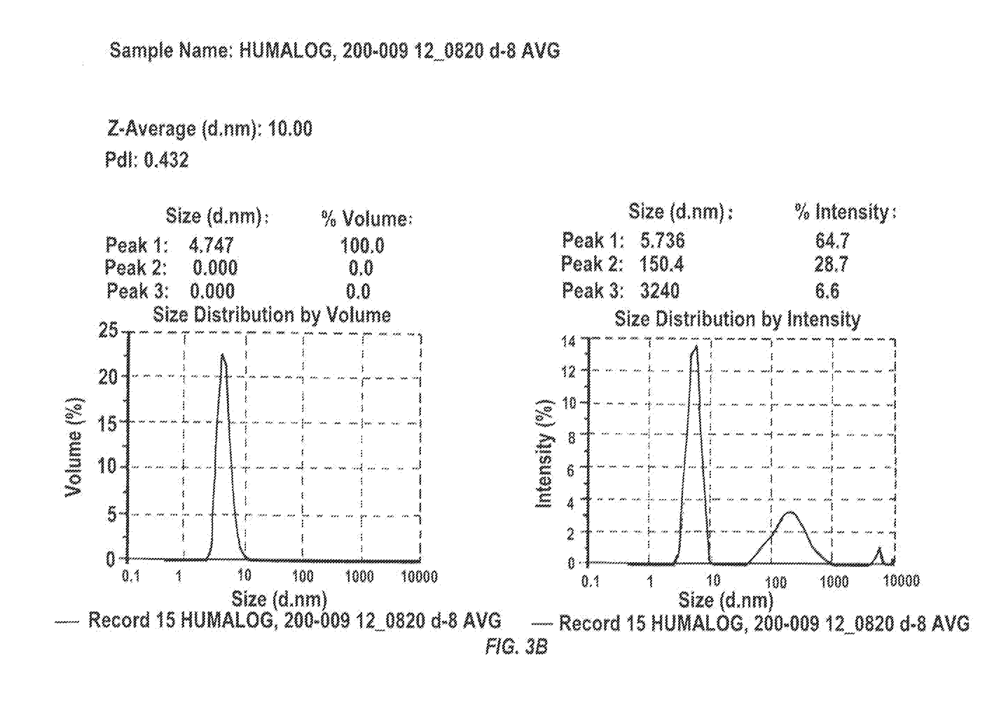

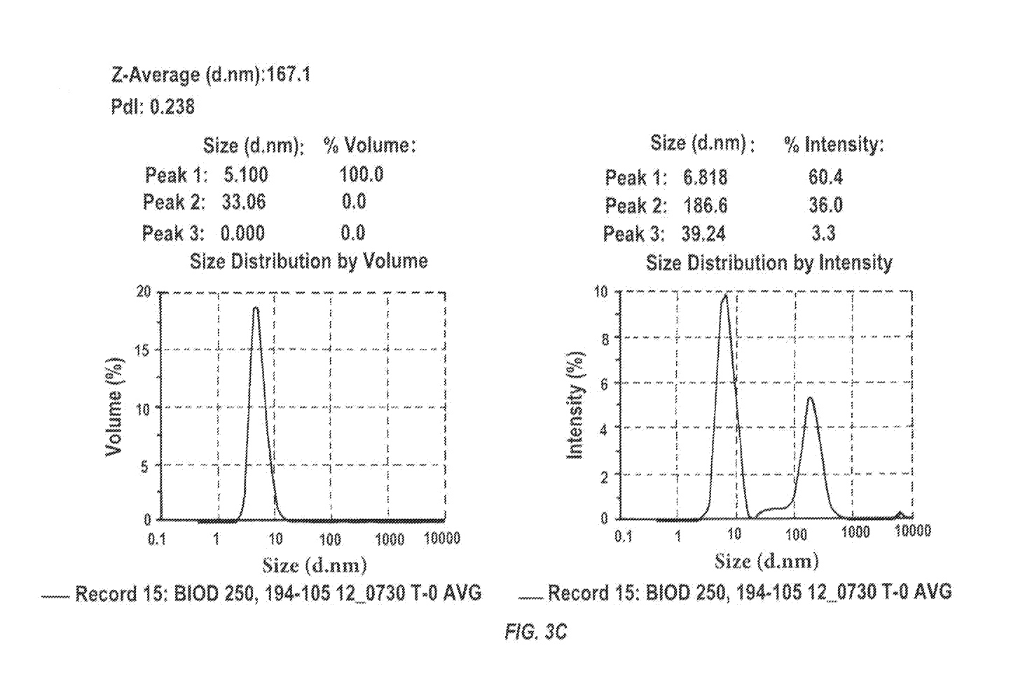

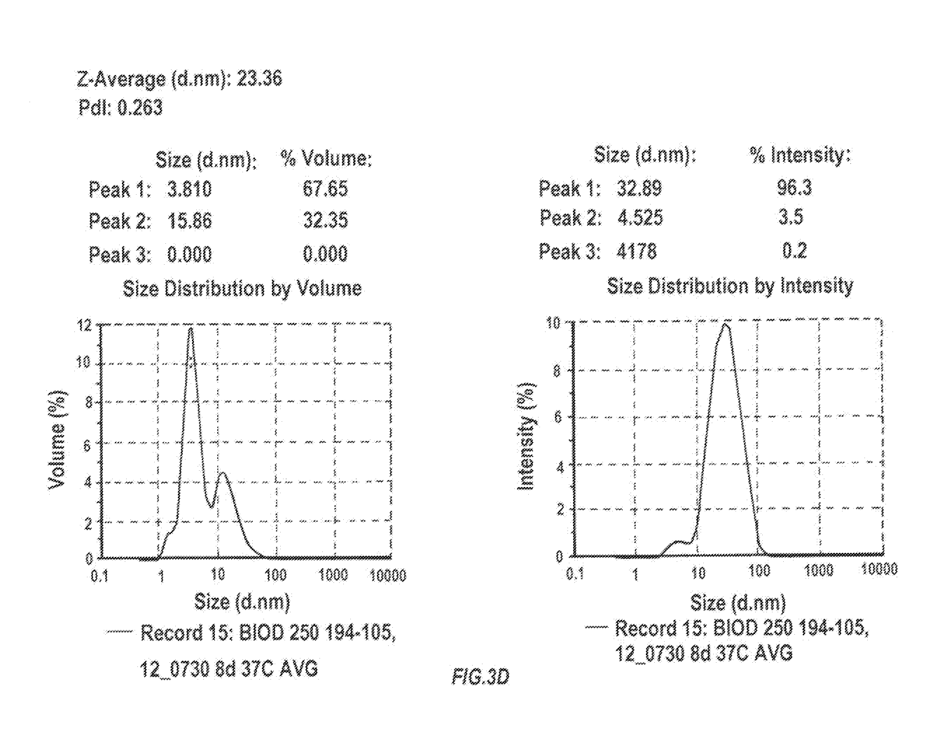

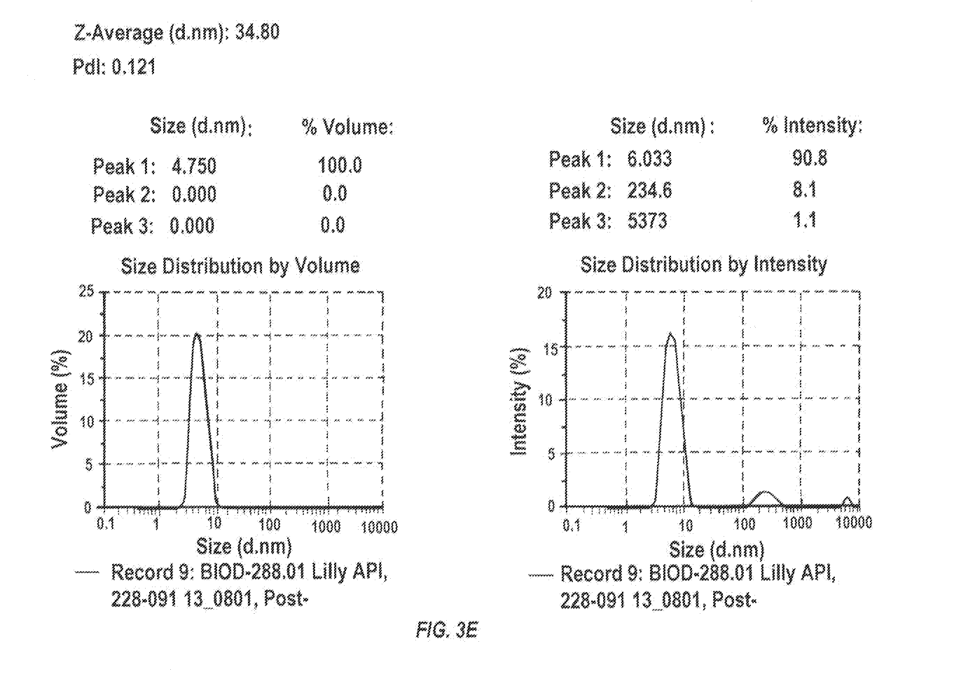

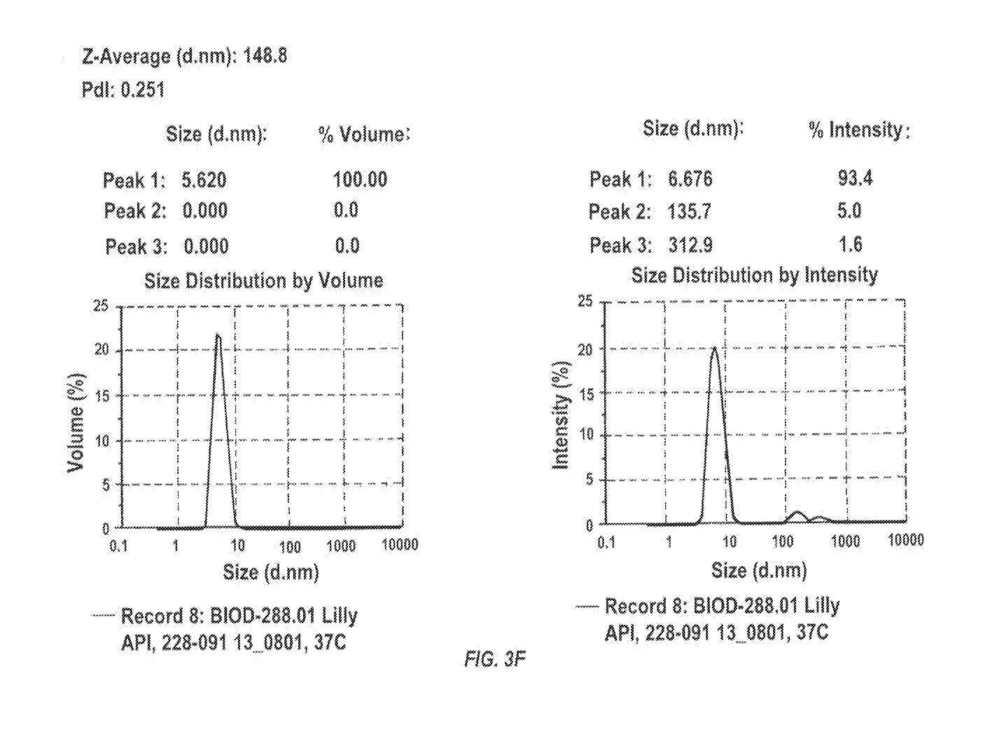

Dynamic light scattering analysis was performed on the following formulations: HUMALOG®, BIOD-250, BIOD-286, BIOD-288, BIOD-290 and BIOD-294. The baseline measurements (FIGS. 3A (HUMALOG®), 3C (BIOD-250), 3E (BIOD-290), BIOD-286, BIOD-288, and BIOD-294 were compared to the measurements at day 7, at 37° C. (FIGS. 3B (HUMALOG®), 3D (BIOD-250), 3F (BIOD-290), BIOD-288, and BIOD-294, or day 13 at 37° C. (BIOD-286). The size distributions of BIOD-250 progressively change, showing a population of smaller and larger size insulin particles. These data are consistent with a formulation that initially starts as a hexamer, then dissociates into monomers and dimers.

The size distributions of BIOD-290 at baseline and 13 days at 37° C. are comparable to HUMALOG® at baseline and 7 days at 37° C., respectively. The size distributions of BIOD-286, BIOD-288 and BIOD 294 at baseline and 7 days at 37° C. are also comparable to HUMALOG® (data not shown).

### Example 9

### Physical Characterization Studies of HUMALOG®, BIOD-250, BIOD-286, BIOD-288, BIOD-290 and BIOD-294 Using Analytical Ultracentrifugation

Sedimentation velocity analysis (analytical ultracentrifugation) was conducted at 20° C. and 55,000 RPM using interference optics with a Beckman-Coulter XL-I analytical ultracentrifuge. Double sector synthetic boundary cells equipped with sapphire windows were used to match the sample and reference menisci. The rotor was equilibrated under vacuum at 20° C. and after a period of ˜1 hour at 20° C. the rotor was accelerated to 55,000 RPM. Interference scans were acquired at 60 second intervals for 6 hours. The sedimentation coefficient, S, is expressed in terms of the standard solvent (water) at 20° C. S(20,w) is influenced by the density of the solvent and the solution viscosity and can be related to molecular weight.

The model independent sedimentation coefficient distribution g(s) is computed from the concentration distribution across the boundary. Plotting g(s) vs. the sedimentation coefficient provides information about the distribution of molecules with reference to the sedimentation coefficient. Insulin hexamers have a sedimentation coefficient of ˜3 Svedbergs, while monomeric insulin is ˜1 Svedberg. Dimers, trimers and tetramers are between these values.

Insulin hexamers are the most stable form, however are very slowly absorbed. Post injection, there is dilution of the insulin bolus with extracellular fluids, which reduces the concentration of the insulin and allows it to dissociate into smaller subunits. The more rapid the dissociation into subunits corresponds to faster insulin absorption. The goal of these formulations is to create hexameric insulin for enhanced shelf life stability, which rapidly dissociates into dimers/monomers on dilution, creating an ultra-rapid absorption profile.

The curves on the g(s) vs. S distributions show the effect of dilution on the dissociation of insulin. A second graphic representation of the data is presented as c(s) Continuous sedimentation coefficient distribution vs. S. The c(s) distribution plots are sharpened, relative to other analysis methods, because the broadening effects of diffusion are removed by use of an average value for the frictional coefficient. These sharpened peaks over various S values give an idea of what species are present in solution. However, with interacting species, quantitation of the species is not possible.

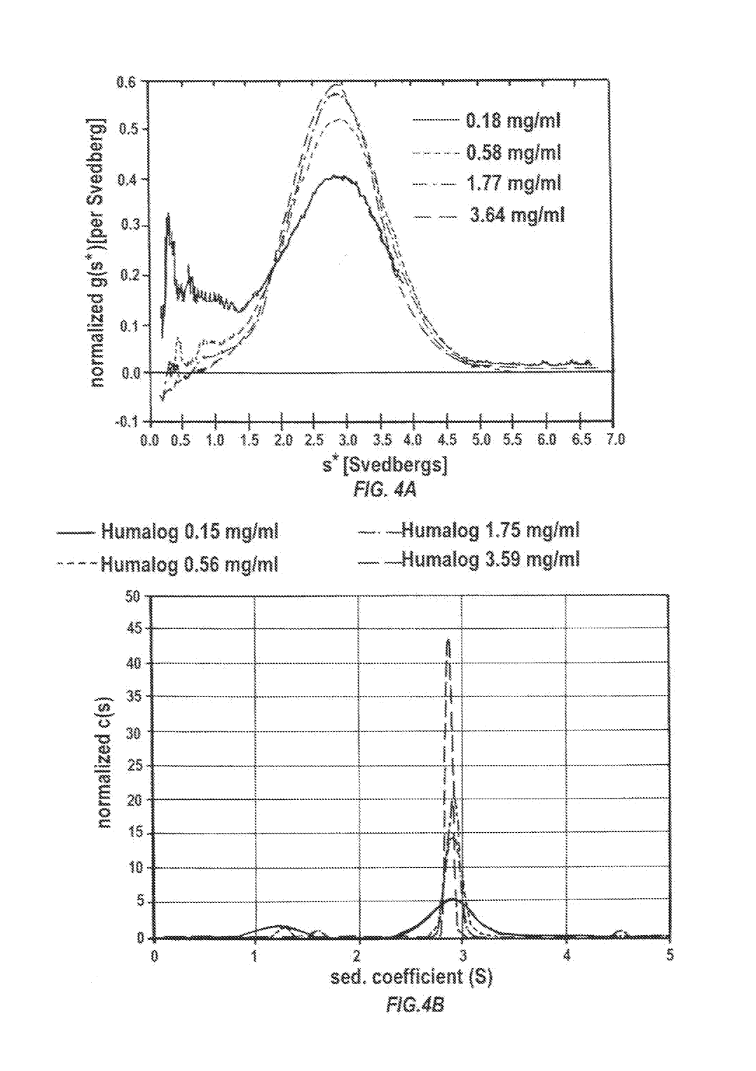

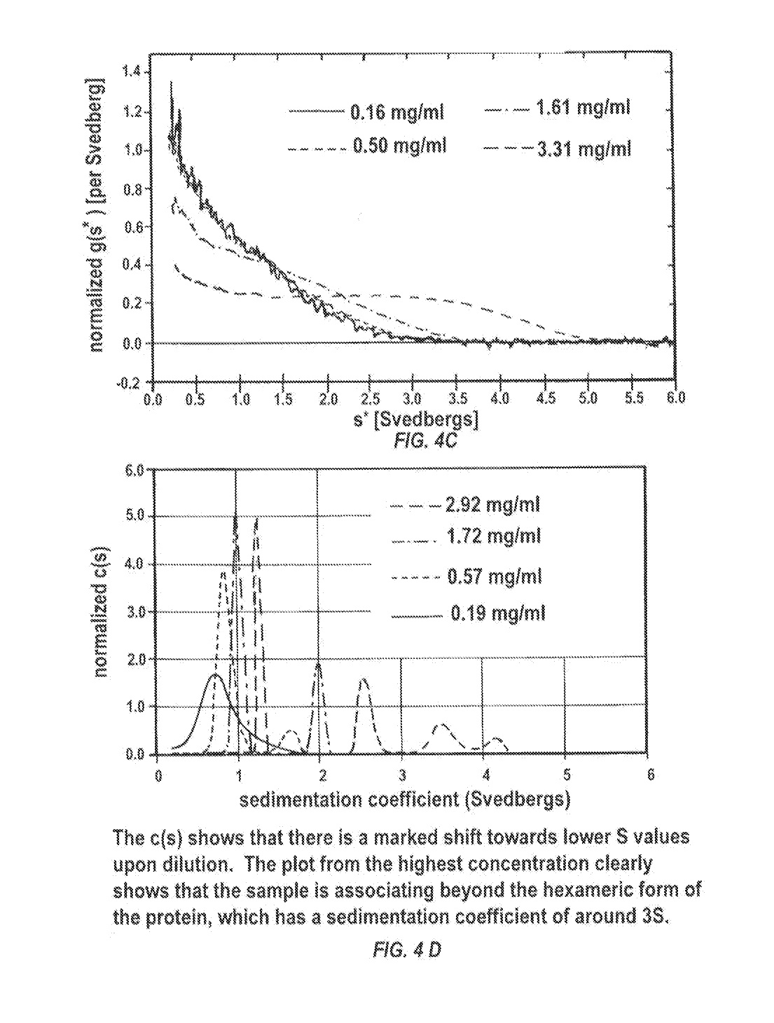

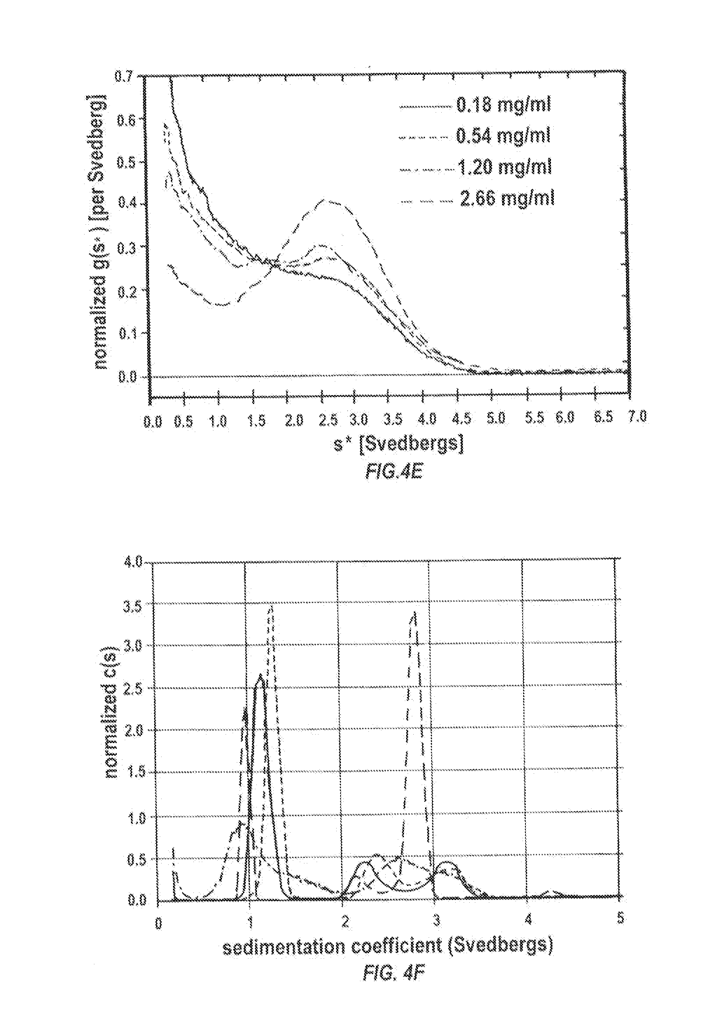

The data is shown for HUMALOG® (FIGS. 4A and 4B), BIOD-250 (FIGS. 4C and 4D), and BIOD-290 (FIGS. 4E and 4F). The data shows that HUMALOG® exits as hexamer suggesting good stability “in the vial or cartridge”. By contrast, BIOD-250 exists as monomers/dimers explaining lack of optimal stability “in the vial”. BIOD-250 exists in many forms including monomers resulting in ultra-rapid absorption. Like HUMALOG®, BIOD-286 (data not shown), BIOD-288 (data not shown), BIOD-290 and BIOD-294 (data not shown) exist primarily as hexamers in the undiluted state suggesting good stability “in the vial or cartridge”.

However, BIOD-290 (but not HUMALOG®) has more monomers prior to dilution and rapidly converts to monomers upon dilution suggesting mechanism for more rapid absorption following subcutaneous administration. BIOD-286, BIOD-288 and BIOD-294 (but not HUMALOG®) display similar characteristics (data not shown). HUMALOG® only converts to monomers at highest dilution resulting in slower absorption.

### Example 10

### Physical Characterization Studies Using Thioflavin T Determination of Fibril Formation

Kinetic Thioflavin Assay (Th T) is used to analyze physical stability of a research or commercial formulation. This method is designed to investigate fibril formation of formulations in its undiluted form with relative low Th T concentration under agitation at accelerated temperatures 45° C., and estimate the fibril formation lag time in the formulations. The estimated Lag time is determined by taking the cross point of baseline and linear slope of fluorescence at dramatic rising phase. The methodology allows for high throughput screening method using a 96 well plate format. The test uses stress conditions which are at 45° C. with agitation over 16 hour run time.

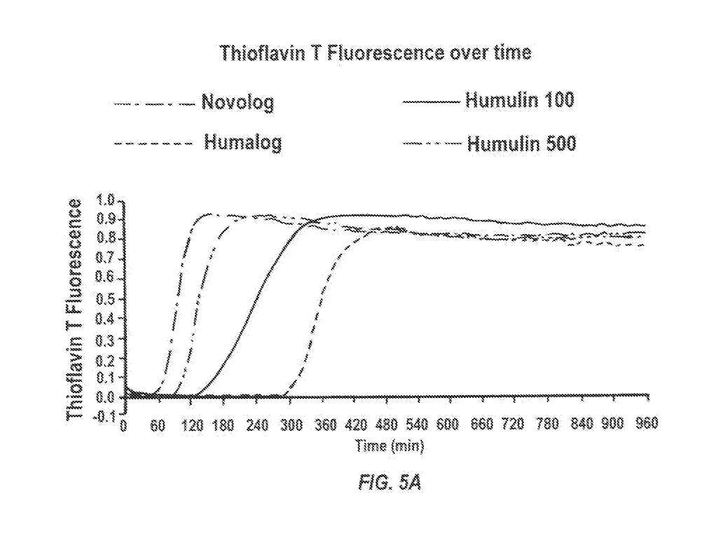

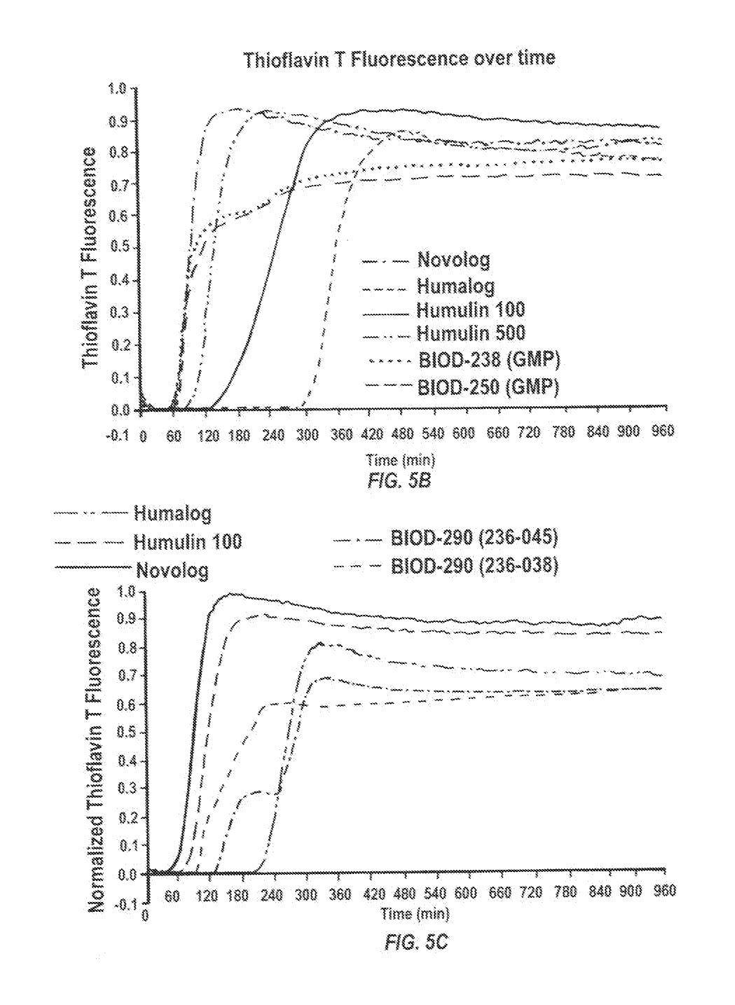

The Thioflavin T fluorescence overtime for NOVOLOG®, HUMALOG®, HUMULIN® 100 and HUMULIN® 500 is shown in FIG. 5A. FIG. 5B shows measurement of kinetic Fibril Formation Profile in a Thioflavin T Assay of BIOD-238 and BIOD-250 Vs. NOVOLOG®, HUMULIN® U-100, HUMULIN® R U-500 and HUMALOG®. FIG. 5C shows the kinetic fibril formation of BIOD-290 vs. NOVOLOG®, HUMULIN® U-100, and HUMALOG®.

These studies indicate that two commercially available formulations HUMALOG® and NOVALOG® have different ThT lag times. As both have acceptable shelf life, it is hypothesized that formulations with lag times that are equal to, or longer than NOVALOG® (ThT lag time shorter than HUMALOG®) should have a commercially acceptable rate of fibril formation. The new BIOD formulations BIOD-286, 288, 290 and 294 have ThT lag times longer or equivalent to NOVALOG®, therefore, should exhibit acceptable shelf life with regard to fibril formation. BIOD-290 data shown for example in Figure Sc, BIOD-286, 288 and 294 have similar acceptable lag times (data not shown).

### Example 11

### Study of Insulin Formulations in Diabetic Swine

Cross over study design: Ten male diabetic miniature swine (35-50 kg) were split into groups corresponding to the number of test articles in each study. On the morning of the study, swine were fasted and 4 pre-dose plasma samples were drawn prior to s.c. dosing of 0.25 U/kg test insulin formulation. Immediately post dose, animals were returned to their pens and fed 500 g swine diet. Subsequent blood samples were pulled at 5, 10, 15, 20, 30, 45, 60, 75, 90, 120, 150,180, 240, 300 and 360 min. post dose. Following a 2-7 day interval, formulations were rotated through the groups using a cross-over study design. Insulin was assayed using a Mercodia iso-insulin ELISA (enzyme-linked immunosorbent assay) and glucose concentration by YSI glucose measurement. (Yellow Springs Instrument).

A summary of absorption, decline from peak and bioavailability characteristics of Lispro-based ultra-rapid-acting insulin formulations in diabetic Swine is shown in Table 9.

TABLE 9
Absorption, decline from peak and bioavailability characteristics of Lispro-based ultra-rapid-acting insulin formulations

| | | BIOD-238 | BIOD-250 | HUMALOG ® | BIOD-286 | BIOD-288 | BIOD-290 | BIOD-294 |
|---|---|---|---|---|---|---|---|---|
| | Variable | N = 19 | N = 20 | N = 60 | N = 20 | N = 30 | N = 29 | N = 20 |
| Absorption | Early ½ Tₘₐₓ | 12 ± 1* | 11 ± 1* | 28 ± 2 | 28 ± 4 | 20 ± 1* | 15 ± 1* | 18 ± 2* |
| | (minutes) | [12] | [11] | [25] | [22] | [16] | [13] | [15] |
| | | (57%↑) | (61%↑) | | (0%) | (28%↑) | (46%↑) | (36%↑) |
| | Tₘₐₓ | 39 ± 8* | 38 ± 6* | 64 ± 4 | 67 ± 6 | 47 ± 4* | 44 ± 5* | 52 ± 8 |
| | (minutes) | [30] | [30] | [60] | [68] | [45] | [30] | [45] |
| | | (39%↑) | (40%↑) | | (5%↓) | (27%↑) | (31%↑) | (19%↑) |
| | AUC_{ins0-30 min} | 2588 ± 227* | 2565 ± 224* | 1344 ± 129 | 1377 ± 158 | 2064 ± 158* | 2159 ± 171* | 1838 ± 183* |
| | (mU*min/L) | [2496] | [2615] | [1068] | [1269] | [1900] | [2003] | [1521] |
| | | (93%↑) | (91%) | | (2%↑) | (54%↑) | (61%↑) | (37%↑) |
| Decline | Late ½ Tₘₐₓ | 99 ± 8* | 110 ± 11 | 122 ± 5 | 132 ± 10 | 107 ± 5 | 118 ± 7 | 116 ± 8 |
| from peak | (minutes) | [91] | [101] | [118] | [136] | [107] | [120] | [113] |
| concen- | | (19%↑) | (10%↑) | | (8%↓) | (12%↑) | (3%↑) | (5%↑) |
| tration | AUC_{ins120-360 min} | 6307 ± 908* | 6496 ± 672 | 6954 ± 440 | 6943 ± 451 | 6201 ± 582 | 6909 ± 719 | 6109 ± 673 |
| | (mU*min/L) | [5404] | [6049] | [6907] | [6944] | [5134] | [5711] | [5158] |
| | | (9%↑) | (7%↑) | | (0.2%↑) | (11%↑) | (1%↑) | (12%↑) |
| Bioavail- | AUC_{ins0-360 min} | 16197 ± 1670 | 16205 ± 1240 | 15583 ± 742 | 15747 ± 1052 | 16008 ± 626 | 16612 ± 1280 | 14643 ± 1175 |
| ability | (mU*min/L) | [15635] | [16751] | [15066] | [15848] | [15021] | [15287] | [15147] |
| | | (4%↑) | (4%↑) | | (1%↑) | (3%↑) | (7%↑) | (6%↓) |

Data represent the Mean ± SEM [median]
*p < .05;
↑ and ↓ represent improvements or decreases Vs. HUMALOG ®, respectively.

A summary of pharmacodynamic parameters of Lispro-based ultra-rapid-acting Insulin formulations in Diabetic Swine is presented in Table 10.

TABLE 10
Pharmacodynamic parameters of Lispro-based ultra-rapid-acting Insulin formulations

| | BIOD-238 | BIOD-250 | HUMALOG ® | BIOD-286 | BIOD-288 | BIOD-290 | BIOD-294 |
|---|---|---|---|---|---|---|---|
| Variable | N = 19 | N = 20 | N = 60 | N = 20 | N = 30 | N = 29 | N = 20 |
| Blood Glucose | −213 ± 17* | −288 ± 10 | −258 ± 9 | −237 ± 18 | −270 ± 14 | −267 ± 11 | −283 ± 13 |
| Minimum | [−222] | [−284] | [−264] | [−246] | [−281] | [−261] | [−295] |
| (BGₘᵢₙ) (mg/dL) | | | | | | | |
| Time to BGₘᵢₙ | 58 ± 8* | 68 ± 7* | 102 ± 5 | 102 ± 10 | 84 ± 5* | 82 ± 6* | 95 ± 8 |
| (minutes) | [45] | [60] | [90] | [105] | [83] | [90] | [83] |
| Time to 50 mg/dL | 18 ± 1* | 18 ± 1* | 32 ± 2 | 25 ± 2 | 21 ± 1* | 22 ± 2* | 24 ± 1* |
| Glucose Reduction | [19] | [17] | [28] | [23] | [20] | [21] | [25] |
| (minutes) | | | | | | | |

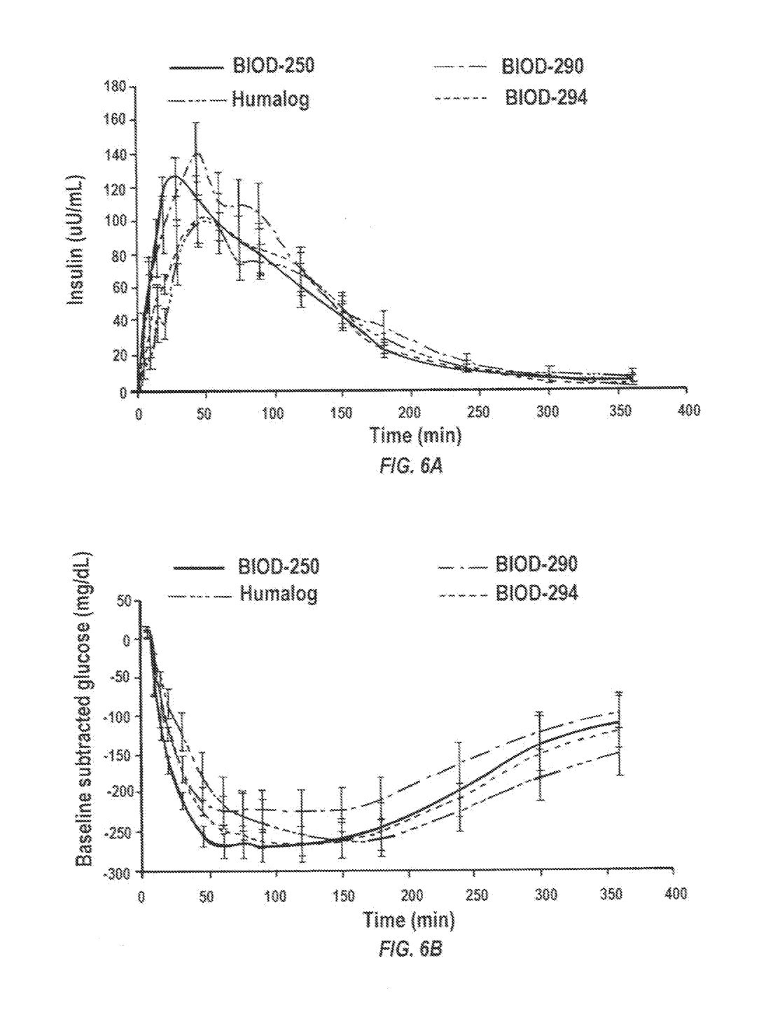

An example of blood insulin levels as a function of time in diabetic swine, following administration of different insulin formulations is shown in FIGS. 6A (BIOD-250, BIOD-290 and BIOD-294 and Humalog). The blood glucose levels (baseline subtracted) are shown in FIGS. 6B (BIOD-250, BIOD-290 and BIOD-294 and Humalog). BIOD-288 also shows an enhanced absorption profile (data not shown).

More rapid absorption as demonstrated by the early ½Tmax and time to glucose reduction can be achieved by reducing the amount of zinc in the formulation.

Summary

BIOD-286, BIOD-288, 290 and 294 show improved stability (relative to BIOD-238 and BIOD-250) and rapid-acting PK and PD profiles in diabetic swine. Mechanisms for improved stability suggest that BIOD-286, BIOD-288, BIOD-290 and 294 like HUMALOG®, is primarily a hexamer “in the bottle” but, has a much faster disassociation to monomers/dimers upon dilution than HUMALOG® explaining the faster absorption following subcutaneous administration. Addition of small amount of zinc and/or “order of addition” in making formulation from lispro API (active pharmaceutical ingredient) may contribute to stability profile and effect the rapidity of absorption in swine.

### Example 12

### Enhanced Stability of Insulin Aspart Formulations

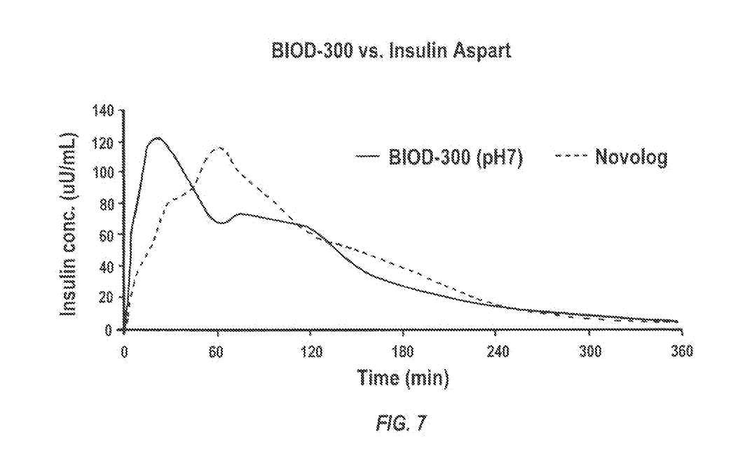

Insulin aspart absorption may also be accelerated using EDTA and citrate formulations. The absorption of insulin in NOVOLOG® is compared to the absorption of insulin aspart in combination with EDTA and citrate (BIOD-300, FIG. 7).

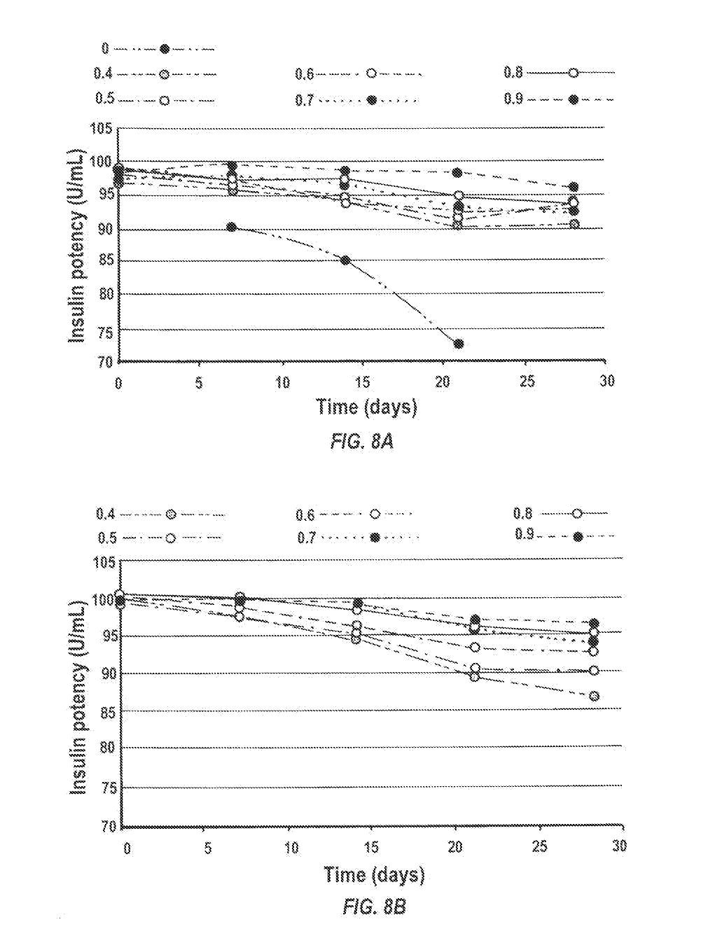

Stability studies were conducted to determine the effect of zinc:insulin hexamer ratio on insulin stability in formulations including insulin aspart. The original formulation (BIOD-300) had a zinc:hexamer ratio of 0. Formulations were prepared by increasing the zinc to hexamer ratios to 0.4, 0.5, 0.6, 0.7, 0.8 and 0.9 and were monitored for insulin potency over time at 37° C. FIG. 8A shows the improvement in stability over BIOD-300 (zinc:hexamer ratio “0”) in formulations using NOVOLOG® as the insulin aspart source. FIG. 8B shows the same formulation ratios using insulin aspart as the API. The data shows that increasing the zinc:hexamer ratio improves the stability of insulin as measured by insulin potency over time.

We claim:

1. An injectable insulin formulation comprising an effective amount of a dissolution/stabilizing agent and an effective amount of a chelator to enhance the stability of the insulin as measured by loss of insulin potency of less than 5 IU at 7 days at 37° C.

2. The formulation of claim 1 wherein the insulin is human recombinant insulin.

3. The formulation claim 1 where the insulin is an insulin analog.

4. The formulation of claim 1 wherein the insulin concentration is 100, 200, 400 or 500 U/mL.

5. The formulation of claim 1 wherein the chelator is EDTA and the concentration of EDTA is between 0.1125 and 0.225 mg/ml of the formulation.

6. The formulation of claim 5, comprising sodium citrate at a concentration between 0.5 and 4.8 mg/ml of the formulation.

7. The formulation of claim 1 comprising a zinc compound providing a zinc:insulin hexamer ratio between 0.4 and 2.6.

8. The formulation of claim 7 wherein the zinc:insulin hexamer ratio is between 0.7 and 0.9.

9. The formulation of claim 6, wherein the dissolution agent is citrate or sodium citrate at a concentration between 0.6 mg/ml and 2.4 mg/ml of the formulation.

10. The formulation of claim 1, further comprising a nicotinic compound in a range between 25 mM and 250 mM.

11. The formulation of claim 10 comprising about 50 mM nicotinamide.

12. The formulation of claim 1, further comprising one or more magnesium compounds are selected from the group consisting of inorganic magnesium salts, organic magnesium salts, and combinations thereof.

13. The formulation of claim 12, wherein the one or more magnesium compounds are Mg(OH)₂, MgSO₄, magnesium EDTA, or combinations thereof.

14. The formulation of claim 1, wherein the concentration of the one or magnesium compounds is about 0.1 to about 10 mg/ml, preferably from about 0.1 to about 5 mg/ml, more preferably from about 0.1 to about 2 mg/ml, most preferably from about 0.2 to about 2 mg/ml.

15. The formulation of claim 1 wherein the dissolution/stabilization agent is selected from the group consisting of acetic acid, ascorbic acid, citric acid, glutamic, succinic, aspartic, maleic, fumaric, adipic acid, and salts thereof.

16. The formulation of claim 1 wherein the dissolution/stabilization agent forms citric ions and the pH is about 7.

17. The formulation of claim 1 further comprising calcium chloride.

18. The formulation of claim 1 further comprising glycerine and m-cresol.

19. The formulation of claim 1, wherein the chelator is sodium EDTA.

20. A method of treating a diabetic individual comprising injecting into the individual an effective amount of the formulations of claim 1.

21. A method of decreasing injection site pain a diabetic individual comprising injecting the individual with an effective amount of the formulation of claim 1.
